(19) United States
(12) Patent Application Publication
(10) Pub. No.: US 2005/0276827 A1
(43) Pub. Date: Dec. 15, 2005

(54) Personal cleansing kit and method having fibrous implement with expiration indicia

(76) Inventors: Filomena Augusta Macedo, Naugatuck, CT (US); Gregory Aaron Grissett, Jacksonville, NC (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 11/000,692

(22) Filed: Dec. 1, 2004

Related U.S. Application Data
(60) Provisional application No. 60/579,592, filed on Jun. 14, 2004.

(51) Int. Cl. A61K 7/50

(52) U.S. Cl. 424/401; 510/130

(57) ABSTRACT

A personal cleansing kit and related method are described. The kit includes a water-insoluble fibrous web folded into a plurality of pleats, each adjacent pleat adhering to one another from about 3 to about 100 separate personal washing events and a cleansing composition having from about 0.5 to about 60% by weight of surfactants in a carrier.

### BACKGROUND OF THE INVENTION

1. Field of the Invention

The invention concerns a kit and method for personal cleansing which includes a disposable fibrous web having indicia to limit usage.

2. The Related Art

Toilet bars and surfactant laden liquids are utilized to cleanse human skin. Copious lather from these products is experientially satisfying and signals a level of cleaning effectiveness.

Implements have long been utilized to enhance speed and volume of lathering. These implements include woven and non-woven textiles, natural and synthetic sponges and loofahs.

U.S. Pat. No. 5,650,384 (Gordon et al.) is typical of the implement technology. Therein disclosed is a hydrophobic diamond-mesh sponge and a liquid cleansing composition packaged together in a kit.

A general problem with implements is microbial contamination. Consumers tend to use, re-use and further use a personal wash implement without consideration that these articles over time breed bacteria, germs and mold. The humid environment in which the implement is used guarantees rapid increase of microbes. Many consumers are unaware of the problem. Others who understand this issue have no convenient way of being reminded of “an expiration date” for the implement.

Clearly there is a need for a mechanism whereby consumers can be alerted that their personal cleansing implement has reached the end of its hygienic life.

### SUMMARY OF THE INVENTION

A personal cleansing kit is provided which includes:
(i) a water-insoluble fibrous web folded into a plurality of pleats, each adjacent pleat adhering to one another for from about 3 to about 100 separate personal washing events;
(ii) a cleansing composition comprising from about 0.5 to about 60% by weight of surfactants in a carrier, wherein each event utilizes at least a foaming amount of the cleansing composition.

Furthermore, the present invention provides a method for cleansing a human body which includes:
(a) providing a cleansing composition which includes from about 0.5 to about 60% by weight of surfactants in a carrier;
(b) contacting a person's body with the cleansing composition;
(c) enhancing lathering of the cleansing composition on the body by applying thereto a water-insoluble fibrous web formed into a plurality of pleats, each adjacent pleat adhering to one another to last from about 3 to about 100 cleansing events.

### BRIEF DESCRIPTION OF THE DRAWING

Various features and advantages of the present invention will become more apparent through consideration of the following drawing in which:

FIG. 1 is a perspective view of a water-insoluble fibrous web according to the present invention;

FIG. 2 is a cross-sectional view of the fibrous web taken along line II-II of FIG. 1; and

FIG. 3 is a cross-sectional view of the fibrous web according to FIG. 2 after a series of continued cleansing events.

### DETAILED DESCRIPTION OF THE INVENTION

Now there is provided a personal cleansing kit and method utilizing a washing implement that provides users with a cue to dispose of the implement after a pre-determined number of washing events. Expiration of the implement avoids extended use of a potentially unhygienic article with microbial growth. The cue is the visual degradation of the implement from a pleated to a relatively non-pleated condition. Further, the relatively non-pleated implement resulting from extended use becomes functionally inferior on foam enhancement and aesthetic feel. The cushion effect of the pleated form degrades to an elongated thin fibrous web of inferior body.

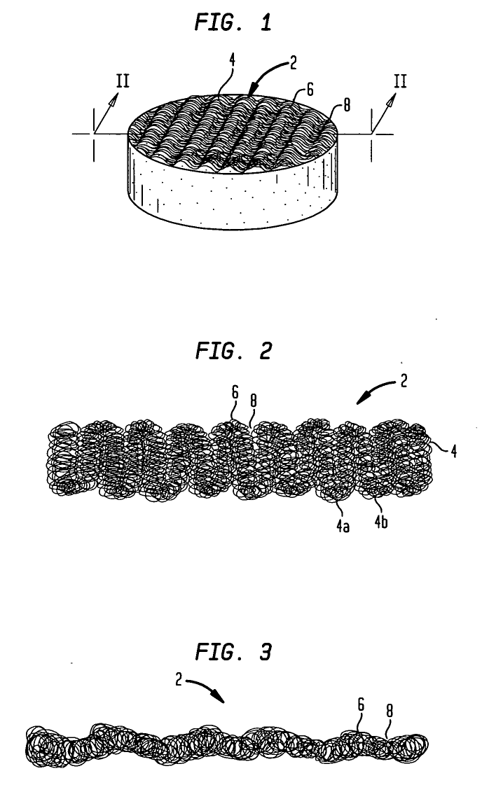

FIG. 1 illustrates one form of a water-insoluble fibrous web implement according to the present invention. Fibrous web 2 is folded into a set of eight pleats 4. Each pleat is characterized by a peak 6 and valley 8.

Adjacent pleats and each fork 4a, 4b of each individual pleat are held together by hydroentangled fibers. Alternatively, adjacent pleats and forks can be temporarily restrained from unfolding by a weak adhesive.

Preferably an applied adhesive will be water-soluble such as a starch or modified starch. Synthetic polymers may also be employed as adhesives, an example of which are the ethylene/vinyl acetate and polyvinyl pyrrolidone polymers.

A total number of pleats may range from about 3 to about 50, preferably from 5 to about 20, optimally from 7 to 10 per implement.

FIG. 2 best shows the pleats 4 along with the fork members 4a, 4b. FIG. 3 illustrates an almost exhausted implement 2 wherein adhesion between adjacent pleats and forks have nearly broken down. Thickness of the overall implement has decreased by more than half of the original thickness.

The webs of the present invention are continuous bonded fiber networks. They are formed of a large number of fiber contact points such that a continuous structure is achieved. The fibers may be synthetic, natural or combinations of these fibers converted via conventional well-known non-woven, woven or knit processing methods. Generally the non-wovens are preferred. Suitable synthetic fibers include but are not limited to polyethylene, polypropylene, polyester, low-melt polyester, viscose rayon, polylactic acid, nylon and any blends/combinations thereof. Additionally, synthetic fibers used herein can be described as staple and continuous filaments. These fibers may be multi-component and have preferably denier ranging from about 1 to about 20 denier. Methods used to arrange and manipulate fibers into a non-woven fibrous web include but are not limited to carding/garnetting, airlay, wetlaid, spunbond, meltblown, vertical lapping or combinations thereof. Cohesion, strength and stability are imparted into the fibrous web via bonding mechanisms such as that of needle punching, stitch bonding, hydroentangling, chemical bonding and thermal bonding and combinations thereof.

Advantageously, fibrous webs of the present invention can range in basis weight from about 25 g/m² to about 1,000 g/m². Lather generating can be improved by proper fibrous web density and porosity. The term porosity (P) can be defined as the volume fraction of air to fibers within a given fibrous web. Porosity can be expressed using the following equation:

$$P = \frac{{P}_{f} - {P}_{w}}{{P}_{f}}$$

wherein P_{f} is fiber density (g/cm³), P_{W} is nonwoven density (g/cm³). Note that the nonwoven density is based on the apparent thickness of the nonwoven structure. Preferably, the fibrous web of the present invention should display porosity ranging from 0.95 to 0.9999.

Another advantageous material property is resiliency. Specifically, Percent Energy Loss is a useful parameter since it describes the resilience of substrates to an applied loss. The Percent Energy Loss is calculated as follows:

$$\% Energy Loss = [ \frac{{J}_{T} - {J}_{R}}{{J}_{T}} ] * 100 ,$$

wherein J_{T}, is the Total Energy required to compress nonwoven to a 100 gram load and J_{R} is the Recovered Energy during one compression cycle. Lower energy loss corresponds to a more resilient nonwoven. Preferably, fibrous webs of the current invention have percent energy loss values ranging from about 5 to about 50%, preferably from about 5 to about 35%.

The test method for Energy Loss involves use of an Instron Tensile/Compression Testing Machine fitted with a 1.5 inch circular die (sample cutting). The compression cycle strain rate is set at 38 mm/min, the recovery cycle strain rate is also set at 38 mm/min. The maximum load is 100 grams load (approximately 0.98 N), the load cell is 5 N, and the platen separation is 31.75 mm. Total energy is measured which is required to compress a sample to 100 grams. Also measured is the recovered energy from one compression cycle. With these two values, the percent Energy Loss can be calculated based on the above equation.

A variety of fibrous webs can be employed for the present invention. Particularly preferred are fibrous batting webs with a continuous network of bonded fibers. In a preferred embodiment, the batting web may have a Loft-Soft Ratio of greater than about 1.1. In other words, the fibrous web of this invention preferably is lofty and fluid-permeable.

As used herein, “lofty” means that the layer has density of from about 0.01 g/cm³ to about 0.00005 g/cm³ and a thickness of from about 0.1 to about 7 cm.

Loftiness of substrates and softness of substrates are related. Softness has several independent, contributing components. One component is a kind of “pillowy” softness. That is, when a force is applied by hand or finger pressure, the substrate easily compresses in much the same way a pillow compresses under pressure to support a body member resting thereon. The web of the present invention is preferably characterized by having a Loft-Soft Ratio of greater than about 1.1, more preferably greater than about 1.3, and most preferably greater than about 1.5.

The methodology for assessing Loft-Soft Ratio is as follows. Substrate samples are cut using a 1.875 inch diameter punch and hammer. In instances where the punching process inelastically compresses edges of discs, the edges are carefully fluffed to restore original dimension. With the top plate in position, the Instron load cell is calibrated and is then run in compression mode at 0.50 inches/minute rate of descent. The Instron may be controlled manually or by computer as long as the final compression is greater than 30 grams/in² pressure and data is collected quickly enough (computer assisted recommended) to determine the height at various compression values during descent. The top plate is then moved down until it contacts the base plate at which point the height is set at zero. It is important that the top plate and base plate are parallel, making contact at all points simultaneously.

Once the apparatus is zeroed, the top plate is retracted to a position above the base plate allowing sufficient space to interpose a substrate sample disc. A substrate disc is then placed in the center of the base plate. The Instron is then set to compress each substrate sample once fully. Next, the Instron is turned on and the height and force of the top plate is continuously recorded. Once the compression of the sample is complete, the compression with new samples of the same substrate is repeated as many times as are needed to establish a reliable average. The average height about the base plate at compression values of 5 gms/in² and 30 gms/in² equals the thickness at 5 gms/in² and 30 gms/in², respectively. The Loft-Soft Ratio is then calculated as the ratio of the thickness at 5 gms/in² divided by the thickness at 30 gms/in².

Personal cleansing compositions of the present invention will contain from about 0.5 to about 60% by weight of the composition of surfactants. Preferably the total weight of surfactants may range from about 5 to about 40%, preferably from about 10 to about 25% by weight of the composition.

One useful surfactant are the fatty acid soaps. The term “soap” is used herein in its popular sense, i.e., the alkali metal or alkanol ammonium salts of aliphatic or alkene monocarboxylic acids. Sodium, potassium, magnesium, mono-, di- and tri-ethanol ammonium cations, or combinations thereof, are suitable for purposes of this invention. The soaps most useful herein are the well known alkalimetal salts of natural of synthetic aliphatic (alkanoic or alkenoic) acids having about 8 to 22 carbon atoms, preferably about 8 to about 18 carbon atoms.

A preferred soap is formed from a saponified mixture of about 30% to about 40% coconut oil and about 60% to about 70% tallow. Mixtures may also contain higher amounts of tallow, for example, 15% to 20% coconut and 80 to 85% tallow.

A second type of surfactant base useful in this invention comprises non-soap synthetic type detergents-so called syndet bases. These may be selected from anionic, nonionic, cationic, amphoteric, zwitterionic and surfactant combinations thereof.

The anionic surfactant may be, for example, a primary alkyl sulfonate, primary alkyl disulfonate, alkene sulfonate, hydroxyalkyl sulfonate, alkyl glyceryl ether sulfonate, aromatic sulfonate, alkyl sulfate, alkyl ether sulfate, alkyl glycerol ether sulfates, alkyl sulfosuccinate, alkyl or acyl taurate, alkyl or acyl sarcosinate, sulfoacetate, alkyl phosphate or phosphonate, alkyl phosphate ester or alkoxy alkyl phosphate ester, acyl lactate, monoalkyl succinate or maleate, acyl isethionate and mixtures thereof. Particularly use are the acyl isethionates such as sodium cocoyl isethionate. Counter cations to the anionic surfactants may be sodium, potassium, ammonium or substituted ammonium such as triethanolammonium and mixtures thereof. Whenever the term alkyl, alkene, aromatic or acyl are employed, this is intended to mean a saturated or unsaturated hydrocarbon of straight or branched chain (or benzenoid type) having from about 6 to about 48 carbon atoms, preferably 6 to 22 carbon atoms.

Zwitterionic surfactants useful for the present invention are broadly described as derivatives of aliphatic quaternary ammonium, phosphonium and sulfonium compounds, in which the aliphatic radicals can be straight or branched chain with from 8 to about 22 carbon atoms.

Amphoteric surfactants useful in this invention may be selected from C₆-C₂₄ betaines, sultaines, hydroxysultaines, alkyliminoacetates, imidoalkanoates, aminoalkanoates, and mixtures thereof. Examples of betaines include coco dimethyl carboxymethyl betaine, coco dimethyl sulfopropyl betaine, oleyl betaine and cocoamidopropyl betaine. Examples of sultaines and hydroxysultaines include materials such as cocoamidopropyl hydroxysultaine. Particularly preferred amphoteric surfactants are cocoamidopropyl betaine, disodium lauroamphodiacetate, sodium lauroamphoacetate and mixtures thereof.

Nonionic surfactants suitable for the present invention are the reaction products of compounds having a hydrophobic group and a reactive hydrogen atom, for example aliphatic alcohols, acids, amides or alkyl phenols with alkylene oxides, especially ethylene oxide either alone or with propylene oxide. Specific nonionic detergent compounds are alkyl (C₆-C₂₂) phenols-ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other so-called nonionic detergent compounds include long chain tertiary amine oxides, long chain tertiary phosphine oxides and dialkyl sulphoxides.

Other nonionics include alkyl glucosides, alkyl polyglucosides, polyhydroxy fatty acid amides, alkoxylated fatty acid esters, sucrose esters, amine oxides and mixtures thereof.

Conditioning agents may be included within the cleansing compositions. These may be selected from such materials as mineral oil, petrolatum, lanolin, lanolin derivatives, C7-C40 branched chain hydrocarbons, C1-C30 alcohol esters of C1-C30 carboxylic acids, C1-C30 alcohol esters of C2-C30 dicarboxylic acids, monoglycerides of C1-C30 carboxylic acids, diglycerides of C1-C30 carboxylic acids, triglycerides of C1-C30 carboxylic acids, ethylene glycol monoesters of C1-C30 carboxylic acids, ethylene glycol diesters of C1-C30 carboxylic acids, propylene glycol monoesters of C1-C30 carboxylic acids, propylene glycol diesters of C1-C30 carboxylic acids, C1-C30 carboxylic acid monoesters and polyesters of sugars, polydialkylsiloxanes, polydiarylsiloxanes, polyalkarylsiloxanes, cyclomethicones having 3 to 9 silicon atoms, vegetable oils, hydrogenated vegetable oils, polypropylene glycol C4-C20 alkyl ethers, di C8-C30 alkyl ethers, and combinations thereof.

Straight and branched chain hydrocarbons having from about 7 to about 40 carbon atoms are useful herein as conditioning agents. Nonlimiting examples of these hydrocarbon materials include dodecane, isododecane, squalane, hydrogenated polyisobutylene, docosane, hexadecane, isohexadecane (a commercially available hydrocarbon sold as Permethyl® 101A by Presperse, South Plainfield, N.J.). Also useful are the C7-C40 isoparaffins. Polydecene, a branched liquid hydrocarbon, is also useful herein and is commercially available under the tradename Puresyn 100® from Mobile Chemical (Edison, N.J.).

Nonlimiting examples of ester type conditioning agents include diisopropyl sebacate, diisopropyl adipate, isopropyl myristate, isopropyl palmitate, myristyl propionate, ethylene glycol distearate, 2-ethylhexyl palmitate, isodecyl neopentanoate, di-2-ethylhexyl maleate, cetyl palmitate, myristyl myristate, stearyl stearate, cetyl stearate, behenyl behenrate, dioctyl maleate, dioctyl sebacate, diisopropyl adipate, cetyl octanoate, diisopropyl dilinoleate, caprylic/capric triglyceride, PEG-6 caprylic/capric triglyceride, PEG-8 caprylic/capric triglyceride, and combinations thereof.

Also useful ester type conditioning agents are various C1-C30 monoesters and polyesters of sugars and related materials. These esters are derived from a sugar or polyol moiety and one or more carboxylic acid moieties. Depending on the constituent acid and sugar, these esters can be in either liquid or solid form at room temperature. Examples of liquid esters include: glucose tetraoleate, the glucose tetraesters of soybean oil fatty acids (unsaturated), the mannose tetraesters of mixed soybean oil fatty acids, the galactose tetraesters of oleic acid, the arabinose tetraesters of linoleic acid, xylose tetralinoleate, galactose pentaoleate, sorbitol tetraoleate, the sorbitol hexaesters of unsaturated soybean oil fatty acids, xylitol pentaoleate, sucrose tetraoleate, sucrose pentaoleate, sucrose hexaoleate, sucrose heptaoleate, sucrose octaoleate, and mixtures thereof.

Nonvolatile silicones such as polydialkylsiloxanes, polydarylsiloxanes, and polyalkarylsiloxanes are also useful conditioning agents. The polyalkylsiloxanes correspond to the general chemical formula R₃SiO[R₂SiO]ₓSiR₃ wherein R is an alkyl group (preferably R is methyl or ethyl) and x is an integer up to about 500, chosen to achieve the desired molecular weight. Commercially available polyalkylsiloxanes include the polydimethylsiloxanes, which are also known as dimethicones, nonlimiting examples of which include the Vicasil® series sold by General Electric Company and the Dow Corning® 200 series sold by Dow Corning Corporation. Also useful are materials such as trimethylsiloxysilicate, which is a polymeric material corresponding to the general chemical formula [(CH₂)₃SiO_{1/2}]ₓ[SiO₂]y, wherein x is an integer from about 1 to about 500 and y is an integer from about 1 to about 500. A commercially available trimethylsiloxysilicate is sold as a mixture with dimethcione as Dow Corning® 593 fluid. Also useful herein are dimethiconols, which are hydroxy terminated dimethyl silicones. These materials can be represented by the general chemical formulas R₃SiO[R₂SiO]ₓSiR₂OH and HOR₂SiO[R₂SiO]ₓSiR₂OH wherein R is an alkyl group (preferably R is methyl or ethyl) and x is an integer up to about 500, chosen to achieve the desired molecular weight. Commercially available dimethiconols are typically sold as mixtures with dimethicone or cyclomethicone (e.g. Dow Corning® 1401, 1402, and 1403 fluids). Also useful herein are polyalkylaryl siloxanes, such as polymethylphenyl siloxanes as SF 1075 methylphenyl fluid (sold by General Electric Company) and 556 Cosmetic Grade phenyl trimethicone fluid (sold by Dow Coming Corporation). Silicones such as methyldecyl silicone and methyloctyl silicone are useful herein and are commercially available from the General Electric Company. Also useful herein are alkyl modified siloxanes such as alkyl methicones and alkyl dimethicones wherein the alkyl chain contains 10 to 50 carbons. Such siloxanes are commercially available under the tradenames ABIL WAX 9810 (C₂₄-C₂₈ alkyl methicone) (sold by Goldschmidt) and SF1632 (cetearyl methicone) (sold by General Electric Company).

Vegetable oils and hydrogenated vegetable oils are also useful herein as conditioning agents. Examples of vegetable oils and hydrogenated vegetable oils include safflower oil, castor oil, coconut oil, cottonseed oil, menhaden oil, palm kernel oil, palm oil, peanut oil, soybean oil, rapeseed oil, linseed oil, rice bran oil, pine oil, sesame oil, sunflower seed oil, borage oil, maleated soybean oil, polycottonseedate, polybehenate and mixtures thereof.

The cleansing compositions of the present invention may optionally include one or more moisturizing agents. Nonlimiting examples of moisturizing agents include those selected from the group consisting of polyhydric alcohols, polypropylene glycols, polyethylene glycols, ureas, pyrrolidone carboxylic acids, ethoxylated and/or propoxylated C3-C6 diols and triols, alpha-hydroxy C2-C6 carboxylic acids, ethoxylated and/or propoxylated sugars, polyacrylic acid copolymers, sugars having up to about 12 carbon atoms, sugar alcohols having up to about 12 carbon atoms, and mixtures thereof. Specific examples of useful conditioning agents include materials such as urea; guanidine; glycolic acid and glycolate salts (e.g., ammonium and quaternary alkyl ammonium); lactic acid and lactate salts (e.g. ammonium and quaternary alkyl ammonium); sucrose, fructose, glucose, erythritol, sorbitol, mannitol, glycerol, hexanetriol, propylene glycol, butylene glycol, hexylene glycol, and the like; polyethylene glycols such as PEG-2, PEG-3, PEG-30, PEG-50, PEG-100, PEG-14M; polypropylene glycols such as PPG-9, PPG-12, PPG-15, PPG-17, PPG-20, PPG-26, PPG-30, PPG-34; alkoxylated glucose; hyaluronic acid; cationic skin conditioning polymers (such as Polyquaternium polymers); and mixtures thereof. Glycerol known also as glycerin, in particular, is a preferred moisturizing agent in the compositions of the present invention.

Cationic polymers may be natural backbone quaternary ammonium polymers selected from the group consisting of Polyquaternium4, Polyquaternium-10, Polyquaternium-24, PG-hydroxyethylcellulose alkyldimonium chlorides, guar hydroxypropyltrimonium chloride, hydroxypropylguar hydroxypropyltrimonium chloride, and combinations thereof; synthetic backbone quaternary ammonium polymers selected from the group consisting of Polyquaternium-2, Polyquaternium-6, Polyquaternium-7, Polyquaternium-11, Polyquaternium-16, Polyquaternium-17, Polyquaternium-18, Polyquaternium-28, Polyquaternium-32, Polyquaternium-37, Polyquaternium-43, Polyquaternium-44, Polyquaternium-46, polymethacylamidopropyl trimonium chloride, acrylamidopropyl trimonium chloride/acrylamide copolymer, and combinations thereof; natural backbone amphoteric type polymers selected from the group consisting of chitosan, quaternized proteins, hydrolyzed proteins, and combinations thereof; synthetic backbone amphoteric type polymers selected from the group consisting of Polyquaternium-22, Polyquaternium-39, Polyquaternium-47, adipic acid/dimethylaminohydroxypropyl diethylenetriamine copolymer, polyvinylpyrrolidone/dimethylaminoethyl methacrylate copolymer, vinylcaprolactam/polyvinylpyrrolidone/dimethylaminoethylmethacrylate copolymer, vinylcaprolactam/polyvinylpyrrolidone/dimethylaminopropylmethacrylamide terpolymer, polyvinylpyrrolidone/dimethylaminopropylmethacrylamide copolymer, polyamine; and combinations thereof.

When the cationic polymer is a polyamine, it is preferred that the cationic polyamine polymer be selected from the group consisting of polyethyleneimines, polyvinylamines, polypropyleneimines, polylysines and combinations thereof. Even more preferably, the cationic polyamine polymer is a polyethyleneimine.

Therapeutic benefit agents may be incorporated into the compositions. Illustrative but not limiting are anti-acne actives, anti-wrinkle actives, anti-microbial actives, anti-fungal actives, anti-inflammatory actives, topical anaesthetic actives, artificial tanning agents and accelerators, anti-viral agents, enzymes, sunscreen actives, anti-oxidants, skin exfoliating agents, and combinations thereof.

Vitamins may be included in the compositions. Illustrative are Vitamin A and derivatives (e.g. beta carotene, retinol, retinoic acid, retinyl palmitate, retinyl linoleate, retinyl acetate), Vitamin B (e.g. niacin, niacinamide, riboflavin, pantothenic acid and derivatives), Vitamin C (e.g. ascorbic acid, ascorbyl tetraisopalmitate, magnesium ascorbyl phosphate), Vitamin D, Vitamin E and derivatives thereof (tocopherol, tocopherol palmitate, tocopherol acetate), and mixtures thereof.

Sunscreens may be incorporated into the compositions. Particularly useful are the benzophenone sunscreens such as benzophenone-4, octyl methoxycinnamate (Parsol MCX) and Avobenzene (Parsol 1789). Amounts of the sunscreen may range from about 0.0001 to about 8% by weight of the foamable composition.

Chelates may also be incorporated into the compositions. Particularly preferred are such chelates as sodium EDTA, phosphates and phosphonates such as Dequest 2010® (EHDP) and mixtures thereof.

The compositions may contain fatty acids which have carbon content from about 8 to about 22. Illustrative fatty acids are stearic acid, palmitic acid, oleic acid, lauric acid, myristic acid, hydroxystearic acid and mixtures thereof. Amounts of the fatty acid may range from about 0.1 to about 40% by weight of the cleansing compositions.

Cleansing compositions of the present invention can contain water as a carrier. Amounts of water may vary from about 1% to about 90%, preferably from about 20% to about 80%, optimally from about 50% to about 70% by weight of the composition.

Compositions of the present invention will generally also contain anti-microbial agents. Illustrative but not limiting examples include methyl paraben, ethyl paraben, propyl paraben, sodium sorbate, sodium benzoate, dimethylol dimethyl hydantoin (DMDM hydantoin), iodopropynylbutylcarbamate, methylchloroisothiazolinone, methylisothiazolinone, trichlosan, trichlorban and mixtures thereof. Amounts of the anti-microbials may range from about 0.0001 to about 2% by weight of the foamable composition.

A wide variety of regulatory approved colorants may be employed. Merely for illustrative purposes these include Red 4, Yellow 5, Blue 1, Titanium Dioxide and mixtures thereof.

Personal cleansing kits according to the present invention advantageously can package the fibrous web together with a bottle filled with flowable cleansing composition. Plastic or cellulosic packaging can enclose these kit components. It may be useful to have a transparent window within the package for viewing of the fibrous web to alert the consumer to the presence and type of this implement. Instructions may be printed onto the packaging, container delivering the cleansing composition or as a separate sheet within the kit on method of using components thereof. The instructions for the method may include the following language: Gently squeeze cleansing product from the container onto the hand, body or directly onto the fibrous web. Apply water to the cleaning composition and rub with the fibrous web until a creamy lather is produced. After cleansing the skin thoroughly, the composition is rinsed clean to leave skin feeling soft and smooth. The fibrous web is allowed to dry between bath, shower or washing events. Once the fibrous web has unfolded or substantially unfolded, it should be discarded.

By the term “washing event” is meant use of the fibrous web against the body for a period from about 1 minute to about 30 minutes. Each event may utilize from about 1 to about 50 grams, preferably from about 3 to about 15 grams of cleansing composition per event.

In the context of the present invention, personal cleansing compositions preferably are in liquid form, flowable at 20° C. Less preferably but within the concept of the present invention are toilet bars as cleansing compositions.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

Except in the operating and comparative examples, or where otherwise explicitly indicated, all numbers in this description indicating amounts of material ought to be understood as modified by the word “about”.

The following examples will more fully illustrate the embodiments of this invention. All parts, percentages and proportions referred to herein and in the appended claims are by weight unless otherwise illustrated.

### EXAMPLE 1

A kit is assembled including a needle punched rayon/polypropylene hydroentangled fibrous web available from Structured Fibers, Inc. in an amount of 2 grams weight. A polypropylene screw-cap container holding a cleansing composition is inserted adjacent the fibrous web into a cardboard carton having a transparent plastic window. The cleansing composition has the formula outlined in Table I below.

TABLE I

| Ingredients | Weight % |
|---|---|
| Titanium Dioxide | 0.10 |
| Preservative | 0.20 |
| Polymer JR 30 | 0.40 |
| Perfume | 1.80 |
| Alkylpolysacharide | 2.00 |
| Maleated Soybean Oil | 2.00 |
| Myristic Acid | 2.00 |
| Coconut Monoethanol Amide | 2.80 |
| Glycerine | 3.00 |
| PEG(6) Caprylic/Caprylglycerate | 4.00 |
| Cocoamphoacetate/Cocoamphodiacetate | 6.00 |
| Soybean Oil | 8.00 |
| Sodium Laureth-3 Sulfate | 12.0 |
| Water | 55.70 |

### EXAMPLE 2

Another kit is assembled. Included is a needle punched polyethylene/Tencel® hydroentangled fibrous web of 2 grams weight available from Structured Fibers, Inc. A polypropylene container with flip-top lid is utilized to hold a cleansing composition. The web and container are inserted adjacent one another into a cardboard carton having a transparent plastic window. The cleansing composition has the formula outlined in Table II below.

TABLE II

| Ingredient | Weight % |
|---|---|
| Benzophenone-4 | 0.05 |
| Ammonium Chloride | 0.15 |
| Methyl Paraben | 0.3 |
| Propyl Paraben | 0.3 |
| Disodium EDTA | 0.4 |
| Fragrance | 0.8 |
| Petrolatum | 1.0 |
| Glycerin | 1.0 |
| Sunflower Seed Oil | 2.0 |
| Ammonium Laureth Sulfate | 8.0 |
| Cocoamidopropylbetaine | 8.0 |
| Ammonium Lauryl Sulfate | 12.0 |
| Water | Balance |

1. A personal cleansing kit comprising:
(i) a water-insoluble fibrous web folded into a plurality of pleats, each adjacent pleat adhering to one another for from about 3 to about 100 separate personal washing events;
(ii) a cleansing composition comprising from about 0.5 to about 60% by weight of surfactants in a carrier, wherein each event utilizes at least a foaming amount of the cleansing composition.

2. The kit according to claim 1 wherein the fibrous web has from 5 to about 20 pleats.

3. The kit according to claim 1 wherein the fibrous web is a hydroentangled needle punched textile.

4. The kit according to claim 1 wherein the fibrous web has a Loft-Soft Ratio greater than about 1.1.

5. A method for cleansing a human body comprising:
(a) providing a cleansing composition comprises from about 0.5 to about 60% by weight of surfactants in a carrier;
(b) contacting a person's body with the cleansing composition;
(c) enhancing lathering of the cleansing composition on the body by applying thereto a water-insoluble fibrous web formed into a plurality of pleats, each adjacent pleat adhering to one another to last from about 3 to about 100 cleansing events.
